(19) United States
(12) Patent Application Publication
(10) Pub. No.: US 2007/0077236 A1
(43) Pub. Date: Apr. 5, 2007

(54) Method for cell implantation

(75) Inventor: Kurt Osther, Scottsdale, AZ (US)

Correspondence Address: EDWARDS & ANGELL, LLP, P.O. BOX 55874, BOSTON, MA, 02205, US

(73) Assignee: Interface Biotech A/S

(21) Appl. No.: 10/560,308

(22) PCT Filed: Jun. 11, 2004
(86) PCT No.: PCT/DK04/00408

§ 371 Date: Nov. 1, 2006

(30) Foreign Application Priority Data
Jun. 12, 2003 (DK) PA 2003 00871

(51) Int. Cl. A61K 35/32; A61B 1/00

(52) U.S. Cl. 424/93.7; 600/102

(57) ABSTRACT

An endoscopic method for treating cartilage or bone defects in an animal, said method comprising the steps of: I) identifying the position of the defect, ii) applying cells selected from the group consisting of chondrocytes, chondroblasts, osteocytes and osteoblasts and combinations thereof into the cartilage or bone defect. In particular, the invention relates to a method for arthroscopic or endoscopic implantation of homologous or autologous cells into a defect of an animal body, the method comprising a step of I) arthroscopic or endoscopic application of a fluid to a cavity or surface containing the defect, and the steps of ii) application of the cells to the defect substantially simultaneously with a support material, the application being performed at the defect covered by the fluid, iii) mixing of the cells and the supporting material, iv) solidification of the supporting material so that the defect is covered by a mixture of cells and support material without any significant amount of fluid, and v) optionally, removal of the fluid from the cavity or surface by drainage or suction.

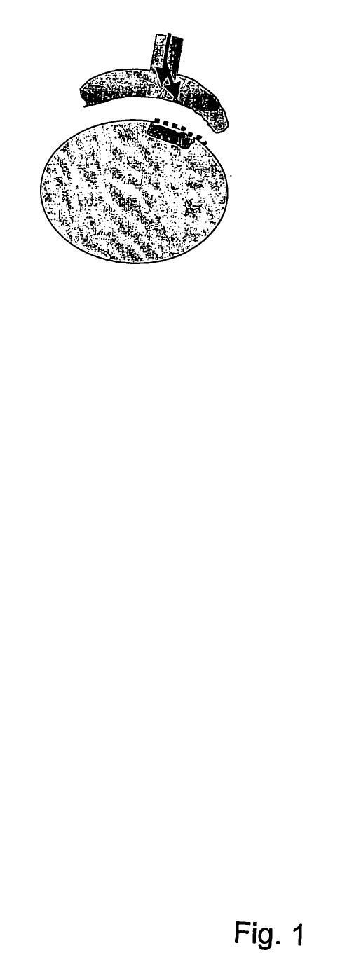

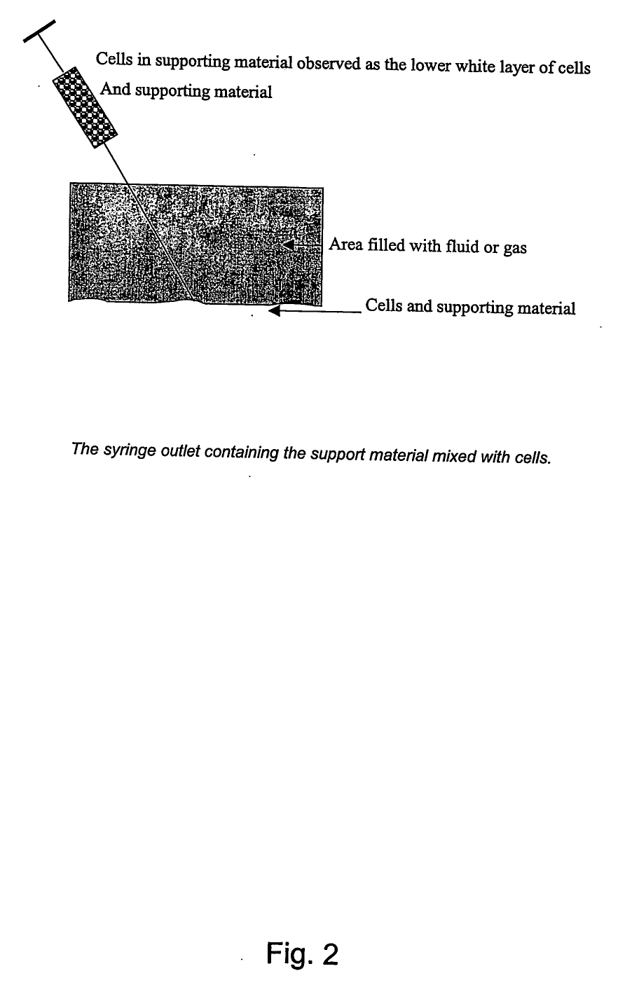

### FIELD OF THE INVENTION

The present invention relates to a method for cell implantation or transplantation involving the use of an arthroscope or an endoscope. The method enables arthroscopic and/or endoscopic implantation of cells and is an easy, safe and cheap method.

### BACKGROUND OF THE INVENTION

Mats Brittberg et al. have shown that a fibrin adhesive such as Tisseel® with, or without Growth hormone does not have a chondrogenic effect on immature chondrocytes, and it is therefore not suitable as a scaffold to promote repair of chondral or osteochondral defects (Brittberg, M, Sjögren-Jansson, Lindahl, A, Peterson, L, The influence of fibrin sealant on Osteochondral Defect Repair in the Rabbit Knee, from Mats Brittberg's Thesis, Cartilage Repair. On cartilaginous tissue engineering with the emphasis on chondrocyte transplantation, Dep. Of Orthopedics, Institute of Surgical Sciences and Dep. Of Clinical Chemistry, Institute of Laboratory Medicine, Göteborg University, Sweden, 1996).

Lazovic and Messner (Lazovic, D, et al., Acta Ortop. Scand, 1993,64:583-586) examined the use of the fibrin adhesive (Tisseel®) in an attempt to improve the healing of transected anterior cruciate ligament in dogs, and found that ligaments, which were repaired using the adhesive, showed low range development of collagen versus simply suturing the ligament, which actually resulted in much higher content of collagen. They postulated that fibrin adhesive chemically enhanced cell proliferation, but the repair tissue formed was of inferior quality. In Brittberg et al's experiments using Tisseel® for the repair of osteochondral defects in rabbits, they found that there was less repair tissue in the Tisseel® group, but the repair tissue was of the same fibrous-fibrocartilaginous quality regardless of whether Tisseel® was used or not used. They concluded that Tisseel® did not improve the repair of osteochondral defect, whereas autologous or homologous fibrin clots used as support appeared to show beneficial effect sufficient to merit further in vivo exams.

Further, Brittberg et al. found that experiments where chondrocytes were used together with Tisseel® did not exhibit any toxic reaction, but the chondrocytes were only growing on the surface of the Tisseel® clot indicating that Tisseel® could be used as a barrier, such as for instance sealing the periphery of a cover such as for instance a periosteal graft as used in ACI. However experiments including homologous fibrin clots showed full in-growth of both rabbit and human chondrocytes, thus indicating that this type of clot showed a “scaffold-conductive” effect on the cells.

In experiments with Tisseel® plus injection of growth hormone (GH) it did not reinforce any scaffolding effect, but would rather serve as a hemostatic barrier, whereas homologous (and autologous) fibrin appeared to promote infiltration of and growth of chondrocytes into the fibrin clot, thus indicating that this type of fibrin may be exhibiting a scaffolding effect. It is therefore possible that homologous (or autologous) fibrin may exhibit a growth promoting effect on chondrocytes and an increased migration into the fibrin.

The development of a suitable arthroscopic method for chondrocyte implantation and more specifically, autologous chondrocyte implantation, as well as for cell implantation methods—besides for the repair of chondral lesions—theoretically to be used for the repair of osteochondritis, osteoarthritis, also called osteoarthrosis, and chondromalacia of patella, etc. would significantly bring the cost down, when compared to ACI (Autologous Chondrocyte Implantation) also by some authors called ACT (Autologous Chondrocyte Transplantation) performed during open knee surgery. Next, chondrocyte implantation and other cell implantations would render the ACI technology as well as other cell implantation technologies in the orthopedic field, more accessible in—besides knee joints, other joints. Further, virtually any orthopedic clinics that performs arthroscopic interventions, would gain access to using the ACI technology, and thereby more patients would be able to be treated with ACI in the repair of their cartilage defects (and later, when cell implantation for other orthopedic diseases becomes available, these techniques would also be subjected to development of arthroscopic guided treatment.

Accordingly, there is a need for developing an arthroscopic method in order to enable a fuller utilization of the cell implantation technology in the field of orthopedic surgery.

Methods using scaffold technologies of various forms, where the scaffold (with, or without cells grown in the scaffold) is inserted into the defect, have suffered from difficulties in performing the cell implantation procedure solely guided by arthroscopy.

### DETAILED DISCLOSURE OF THE INVENTION

The objective of the invention is to provide an arthroscopic ACI treatment method of articular cartilage, bone defects or combination of cartilage and bone (osteoarthritis) in various joints in the human body or in animals. The method provides a technique wherein a cell suspension is applied through a portal (“keyhole”) into the articular joint, leading to sedimentation of cultured cells and adhesion of cells to subchondral bone and/or cartilage. The present invention therefore relates to an endoscopic method for treating cartilage or bone defects in a subject, said method comprising the steps of:
i) identifying the position of the defect,
ii) applying cells selected from the group consisting of chondrocytes, chondroblasts, osteocytes and osteoblasts and combinations thereof into the cartilage or bone defect.

When an arthroscope is used to identify the position of the defect in step i), and used to guide the injection of cultured autologous or homologous cells in step ii), the method is called Arthroscopic ACI.

In the context of the present application and invention the following definitions apply:

Arthroscopic Autologous Cell Implantation (arthroscopic ACI) is a medical procedure for treating cartilage or bone defects, whereby cultured cells are implanted into a defect by a needle as for instance a “blunt” needle or a catheter. This implantation procedure is visualized and guided by an arthroscope.

A suspension is formed when an insoluble component is dispersed in a solvent. In this particular invention, a suspension is formed when the cells to be applied to the defect are mixed with an aqueous medium. The suspension is then applied to the defect. The term “defect area” or “defect cavity” is intended to mean injured articular cartilage, an articular cartilage defect down to and/or involving the bone (osteoarthritis), combination of cartilage and bone defect, or bone which is surrounded by normal cartilage or bone.

An endoscope is a device, which can be used to look at the surface of bone or cartilage tissue without the need for open surgery (i.e. through a portal in the skin of the subject). An arthroscope is a particular type of endoscope, which is used in the examination of joints.

An arthroscope according to the present invention is an instrument (a type of endoscope) which is inserted into a joint for visual examination. An arthroscope usually comprises a tube containing fibre optics, a lens and a light source, and allows joints to be examined without open surgery.

The term “portal” is intended to mean a small operating procedure in which a minor hole is created in the skin in order to introduce an arthroscope as well as arthroscopic devices into articular joint.

The term “cartilage explant” is intended to mean a part of a mammalian articular cartilage, which has been explanted from a mammal by use of a suitable instrument. An explant may contain more than one kind of tissue, e.g., in the case of explantation of tissue from the knee, the explant may contain cartilage tissue as well as bone tissue.

In a specific embodiment, the present invention meets the above-mentioned needs by providing a method for arthroscopic or endoscopic implantation of homologous or autologous cells into a defect of an animal body, the method comprising
i) application of a fluid to a cavity or surface containing the defect via arthroscope or endoscope to locate area of defect to be repaired or target area for cell transplantation,
ii) application of the cells to the defect substantially simultaneously with a supporting material, acting as a binding scaffold, coagulating scaffold or gelating scaffold
iii) mixing of the cells and the supporting material,
iv) solidification of the supporting material so that the defect or the target area is covered by a mixture of cells and supporting material, and
v) optionally, removal of the fluid from the cavity or surface via drainage or suction.

The fluid employed in step i) may be a liquid such as physiologically acceptable a sodium chloride solution, Ringer's solution, a cell culture medium, or the like or it may be a gas such as e.g. sterile air comprising CO₂ in a concentration that is compatible with the tissue in question (e.g. a joint or an organ of the animal body; such as, e.g., a mammal like a human). Normally, a concentration of CO₂ of about 5% is suitable. Such fluids are normally used during arthroscopy and endoscopy and are well known to a person skilled in the art. In a specific embodiment of the invention the fluid is a liquid. The purpose of using the fluid (e.g. the liquid) is to expand the defect in order to enable visualisation thereof by the arthroscope. Accordingly, the fluid does not itself contain the cells which are implanted into the defect. Furthermore, the fluid present in the defect makes it possible to apply the cells below the upper surface of the fluid (i.e. by leading the needle or catheter down into the bottom of the defect and injecting the defect in a manner that the instant gelating or coagulating hits the defect directly, and in this manner applied under the fluid. Moreover, as described below, the fluid is a suitable medium for the mixing of the two required compositions, namely i) the cell, e.g. in the form of a suspension of the cells and ii) the support material e.g. in the form of a composition comprising the support material. As described below, the mixing of the cells with the support material is important in order to in situ immobilise the cells in the defect. Once applied, the support material and the cells must be substantially homogeneous mixed before the support material solidifies or gels and thereby maintains the cells in the support.

In the present context the term “tissue” is used in a broad sense to cover soft tissue (organs etc.) as well as hard tissue (bones, joints etc).

Once the defect is visualized it is possible to apply the cells. The cells are homologous or autologous cells and are of a type that is suitable for use for repairing the defect in question. In the present context the term “homologous cells” is intended to mean that the cells are compatible with the tissue to which they are applied. The term “autologous cells” indicates that the cells are derived from the same subject to which the cells are applied. In the following e.g. the use of chondrocytes and/or osteoblasts (osteocytes) are discussed. The cells applied are normally presented in a suspension, i.e. the cells are suspended in a suitable medium such as, e.g., Dulbecco's Minimal Eagle Medium/F12, DMEM/F12 optionally containing serum (e.g. fetal calf serum or homologous or autologous serum) and/or growth factors, and the time of application, mixed with the supporting material. The cell suspension may be kept together with the medium alone, and thereafter mixed with the ingredients that will form an clot from another injection system; or the cell suspension may be located together with one or more of the ingredients in one chamber, which does not clot the cells in the chamber itself, but after being mixed with the ingredient(s) inducing the gelation or coagulation, coming from the other chamber of the Twin syringe, the mixing starting after the two chambers are connected for instance via a “Y” connection to one single connection, where the gelating or coagulating process is initiated involving the cells and the ingredients, namely when passing through the injection needle or catheter. The following table provides examples of some of the ingredients that might be used, but not limited to.

Content in chamber 1 and chamber 2 of a Twin syringe (or in any other suitable kit for use in a method according to the invention)

| | Range of | | Cells | Range of |
|---|---|---|---|---|
| | ingredients in | Cells contained in | contained in | ingredients in |
| Combinations | chamber 1 | chamber 1 | chamber 2 | chamber 2 |
| Collagen III plus | Collagen III: 0.5-20 mg/ml, | 2 × 10⁵-6 × 10⁶ | Minus cells | *Thrombin |
| other short chain | and | cells pr. ml | | (any origine) |
| collagens | short chains: 0.5-20 mg/ml | | | 0.5 IU to 1000 IU/ml |
| | plus | | | and |
| | | | | CaCl₂, |
| Collagen III plus | Collagen III: 0.5-20 mg/ml, | Minus cells | 2 × 10⁵-6 × 10⁶ | *Thrombin |
| other short chain | and | | cells pr. ml | (any origine) |
| collagens | short chains: 0.5-20 mg/ml | | | 0.5 IU to 1000 IU/ml |
| | plus | | | and |
| | | | | CaCl₂, |
| Fibrinogen, any | Fibrogen: 1-20 mg/ml | 2 × 10⁵-6 × 10⁶ | Minus cells | *Thrombin |
| type or species | | cells pr. ml | | (any origine) |
| And in some cases | | | | 0.5 IU to 1000 IU/ml |
| aprotinin | | | | and |
| | | | | CaCl₂, |
| Fibrinogen, any | Fibrogen: 1-20 mg/ml | Minus cells | 2 × 10⁵-6 × 10⁶ | *Thrombin |
| type or species | | | cells pr. ml | (any origine) |
| And in some cases | | | | 0.5 IU to 1000 IU/ml |
| aprotinin | | | | and |
| | | | | CaCl₂, |
| Fibrinogen, | At a concentration | 2 × 10⁵-6 × 10⁶ | Minus cells | *Thrombin |
| commercially | rate corresponding | cells pr. ml | | (any origine) |
| available | to the amount used | | | 0.5 IU to 1000 IU/ml |
| | in commercial | | | and |
| | Fibrinogen | | | CaCl₂, |
| | products | | | |
| Fibrinogen, | At a concentration | Minus cells | 2 × 10⁵-6 × 10⁶ | *Thrombin |
| commercially | rate corresponding | | cells pr. ml | (any origine) |
| available | to the amount used | | | 0.5 IU to 1000 IU/ml |
| | in commercial | | | and |
| | Fibrinogen | | | CaCl₂, |
| | products | | | |
| *Thrombin of any origine could be from any species, be recombinant, or be prepared as a peptide from thrombin, which has the capability to induce coagulation or gelation; in some cases aprotinin is added as in commercial combinations such as corresponding to Tisseel or in Beriplast. Calcium chloride is added in the amount for instance used in commercial | | | | |
| # combinations. Normally, the concentration of thrombin used is from about 0.5 to about 10 IU, although in some commercial kit the concentration of thrombin is 1000 IU or more. | | | | |

As mentioned above, the cells are applied to the defect substantially simultaneously, preferably simultaneously, with a support material. The application of such a support material is necessary as cells applied in e.g. a liquid as described above may not adhere to the surface of the defect and accordingly, other means may be provided in order to ensure that the cells are contacted with or held in proximity to the defect. The support material is a material that is capable of coagulating or solidifying upon application to the defect. In a preferred embodiment the coagulation, solidification or gelling is brought about by contacting the support material with thrombin or a thrombin-like material optionally in the presence of ions like e.g. calcium or magnesium ions. Thus, the support material should—when it has solidified in situ—function as a tight cover filling out the defect that adhere to the defect or the target area and should enable the cells to be maintained at the defect or target area. Thus, the cells are present not only on the surface of the defect but throughout the whole solidified or coagulated support material, i.e. the cells are present in multi-layers in the defected area. Accordingly, it is possible to apply an amount of cells that is substantially higher than M only the surface of the defect should be covered with a single layer of cells. Furthermore, the cover has the function of protecting the treated defect or target area from the influence from the local environment. An additional layer of cover over the mixed coagulated, clotted (adherent) and/or gelating cell/support material could be, as described in some of the examples below, be even commercially available fibrin sealants such as for instance Tisseel (Baxter) and/or Beriplast (AventisBehring) in order to create an extra sealing cover over the coagulated cell/support, that is covering by filling the entire defect.

In general, the supporting material in which the cells are mixed or dispersed is typically a coagulating, adhering, binding, gelating /or a sealant product. The supporting material may be diluted with a suitable medium before application through a suitable device to the defect or target area. The composition comprising the support material may contain suitable materials as e.g. fibrin, collagen type I, III, II, or the like, and for instance together with Insulin Growth Factor (IGF) and/or other growth factors. Compositions used as collagen fill that may be administered as a solution at present, are those, marketed such as gelatine, collagen type III, collagen type I, collagen type II (under development by Fibrogen, California) as well as short chain collagens ( some of these are commercially available). The coagulating inducer would be as previously described in relation to the upper table, thrombin in one or another form. Within the area of blood products for coagulation it would be any fibrogen (any species, but preferrably human in one or another form), pure or not purified (containing other proteins such as for instance those proteins that are part of the cryoprecipitate); thrombin or any derivate with thrombin-like coagulating, or gelating inducing effect. All those used for this purpose shall be non toxic to the cells.

It is important that the two different liquids (i.e. i) the medium with cells, and ii) the composition comprising the supporting material are applied substantially at the same time. The flow of the supporting material enables the necessary mixing of the cells and material into the defect or target area and ensure an even distribution of the cells in the support material before it solidifies or coagulates. Alternatively or additionally, a flow, fluid or gas, may be applied (e.g. also via the arthroscope) instead.

The invention encompasses the utilization of either implantation of cells simultaneously with a supporting material, into which the cells are mixed during the application,—in the first part of the invention—performed either under a fluid cover, using the general usage of an arthroscope where a fluid is used to keep the joint open for visualization, or as a second part of the invention also in a combination with an arthroscope, which provides a certain pressure of guided sterile air or slight positive pressure to help the surgeon visualize the area under treatment, such as for instance used in other endoscopes—for instance for abdominal exams, etc., where air is supplied under a certain pressure—such as for instance a combination of atmospheric air combined with CO₂, in a concentration well tolerated by the mixed cell/coagulating support material. In this second part of the invention, the “cover” used, under which the mixed cell/coagulation support material may be placed under the air pocket, where the pressure may be around 20 -30 mm Hg.

The third part of the invention relates to a layer of a coagulating substance (support material) which is applied under either the fluid or under the inflated air,—this support material is in itself cell-free but is mixed with the cells in situ before it solidifies

The thickness of the solidified layer depends on the size of the defect and the particular animal species. For humans, the thickness is generally 2-6 mm.

This first part of the invention concerns the utilization of the presence of fluid, the possibility of changing the pressure of fluid in the joint (and possibly sterile air pressure) during a arthroscopically or endoscopically guided procedure combined with controlling the implantation of a liquid substance, mixed with cells, for instance such as chondrocytes for the repair of cartilage defects, by utilizing the pressure to keep the support material, for instance mixed with cells in place, for instance at the bottom of a cartilage defect or other areas of target, while the material admixed with cells—or without the cells the last scenario may happen, when an extra layer of covering fibrin or other non cell containing sealant is placed over the cell/support coagulate, clot or gel. This method utilizes the difference in solubility of of the support material in two different media, namely i) the fluid applied to the defect in order to visualize the defect (fluid cover) and ii) the medium used to apply the support material via the arthroscope. The support material is chosen so that it solidifies or coagulates and adhere to the defect in spite of the presence of the fluid used as fluid cover.

Application of a pressure controls the localization of a composition comprising the support material without mixing with the fluid cover. In other words, the support material or the composition comprising the support material is not soluble in the adjacent fluid present in the joint during the application, i.e. the support material will be maintained in the application area (e.g. the bottom of a joint) where it solidifies and adheres to the application area. The simultaneous application of the cells enables the supporting material to incorporate or envelope the cells so that the cells are maintained at the application area as well. The cells will within the next 48 hours or longer start to produce matrix themselves, and this matrix will eventually both aid to the adherence and later on be the adherent cartilage matrix, and the support material will most probably be absorbed, after a shorter or longer time interval, eventually.

This method combining the presence of fluid or increased pressure, alternatively as a positive pressure (when compared to the pressure in the joint), provides sufficient vizualisation for the surgeon to perform the application of the cell/support material, the fibrin sealant material (for instance commercial product), containing no cells, but overlayering the cell/support material preventing the applied material from leaking into the joint, the decrease in the pressure and/or the removal—of the pressure, when the solidification and adherence is sufficient to hold the implant in place in the defect is a novel technology, while applying the substance with—or without cells, and to alter the pressure of the fluid (or if air gas is used to alter the pressure of the gas), as needed, without exceeding a pressure that would endanger implanted cells. The ability of certain fibrins to allow proliferation and migration of chondrocytes is described by Mats Brittberg, as described above. However, this procedure done by Brittberg, is not performed as a “two phase” system, e.g. under fluid or gas used for visualization.

Another aspect of the invention is that the method described comprising placing a cell mixed in a supporting material under fluid or under air pressure also may be used for application of more than one cell type such as chondrogenic cells (chondrocytes/chondroblasts or other chondrogene cells) and osteogenic cells such as osteoblasts, osteocytes, or other cells, etc. in order to enable a two step procedure, where the bottom part of an osteoarthritic lesion, for instance, initially is treated with a mix of osteogenic cells and supporting material performed under fluid and/or under air pressure,—next a mix of chondrogenic cells and supporting material are layered over the first cell/supporting material (layer 1)—under fluid and/or air pressure.

Yet another aspect of the inventions is to deliver a hydroxyl apatite granulate (e.g., Bio-Oss, Geistlich Biomaterials, Wolhusen, Switzerland) either containing cultured osteogenic cells or delivered together with osteogenic cells—all in a mixture applied instantly together with supporting material under fluid and/or under air pressure in a defect such as an osteoarthritic defect in a joint. Next, at layer of a mixture of chondrogenic cells/supporting material is layered over the bottom layer consisting of hydroxl apatite granulate.

In any the above described parts of the invention in which a mixture of cells and suitable supporting material, or cells, hydroxyapatite, and supporting material is placed—for instance during or via arthroscopic intervention, a cover layer is a cell-free layer of fibrin as for instance Tisseel® biological glue (in which the chondrocytes for instance will not migrate into, which in this case will coagulate with little or no cells in the upper covering layer—will be placed. Both during the presence of positive pressure in the joint or without the positive pressure, as long as the surgeon has substantial overview over the area, where he is applying the sealent.

The cover layer of either fibrin such as the Tisseel® composite may be mixed with another protein (e.g., collagen (for instance collagen type 1) as a soluble substance mixed with fibrin or with Tisseel®, in order to strengthen the cover which will face the surface of the joint tangential to the surrounding healthy cartilage. This may be done during arthroscopy for instance under fluid or air pressure.

The cover may also be a membrane, cut to the size of the defect (e.g., a collagen type I/III membrane) with the side, facing the cell/coagulation support material, coated with a fresh highly concentrated homologous or supporting material or with fresh Tisseel®, in order to prevent the cell/coagulation support material to allow cells into the cover, and at the same time fasten to the cell/coagulation support material. The membrane may also by itself adhere to the cell/coagulation support material.

When using an arthroscopic method It is therefore necessary to be capable of performing application of chondrocytes such as autologous chondrocytes together with a support material, where one is able to control the application of the chondrocytes in a support material that will remain (adhere) to the area in which the cells shall be kept, in spite of the presence of (sterile) physiological saline or nutrient medium in the joint, due to the use of an arthroscope as guidance for performing autologous chondrocyte implantation.

The method developed by the present inventor is considered novel, due to the new concept of placing a chondrocyte-containing substance at a controlled area in for instance a cartilage defect, such as a chondral defect, an osteochondral defect, or even when applying cells such as osteoblasts/osteocytes in a substance such as for instance hydroxyl-apatite, as a thin bottom layer in a joint suffering from osteoarthritis, and thereafter being able to apply a second layer of chondrocyte/substance as well as when applying cells through an endoscopic procedure to a target organ or target area, such as for instance liver, heart, etc. The substance shall be of a nature that at the same time is not toxic to the cells, and in case of chondrocytes, the substance applied under fluid, shall not be toxic to the chondrocytes or may even be promoting chondrocyte in- growth and/or in a maturation stage in which the cell also produces the matrix structure, that ends up being the new cartilage.

Different concentration levels of the suitable support material used for the mixture and variation of the coagulation, adhering, and/or gelating time as well as variation of the volume and concentration of cells to be mixed with the supporting material is part of this invention. Furthermore, cells and/or the supporting material may be mixed with adequate growth factors such as for instance IGF-1, which may help maintain differentiated chondrocyte morphology in fibrin (Fortier, L A, et al., Am. J. Vet. Res. 2002, 63(2) 301-305).

As described above an integral part of the invention is also to create a the above described cover either as a surface of fibrin or of Tisseel® or one of these agents applied together-with another protein which may make a smooth surface with a relative stronger tensile strength (such as for instance soluble collagen), or possibly mixed with various constituents, such as for instance soluble collagen, and other substances providing a smooth surface of the repaired cartilage area, etc. by creating a spreading outlet that, when in use will enable the surgeon to apply the fibrin to a convex or a concave joint surface, in which there is a defect as shown in the following figure. During the arthroscopy a temporary perforated membrane may be placed stretching over the surface of the cartilage, lining and limiting the fibrin/cell mix and fibrin cover, when injected below the fluid or below an air pressure. The membrane shall be easily permeable for fluid used (or air inflated) during the arthroscopy. The membrane is then removed from one of the incisions when the fibrin/cell mix and the fibrin cover are coagulated in place.

Other aspects of the invention appear from the following items and the appended claims. Whenever relevant, the details and particulars described herein apply mutatis mutandis to these aspects.

More specifically, the invention relates to the following items:
1. An endoscopic method for treating cartilage or bone defects in an animal, said method comprising the steps of:
i) identifying the position of the defect,
ii) applying cells selected from the group consisting of chondrocytes, chondroblasts, osteocytes and osteoblasts and combinations thereof into the cartilage or bone defect.
2. A method according to item 1 for arthroscopic or endoscopic implantation of homologous or autologous cells into a defect of an animal body, the method comprising a step of
i) arthroscopic or endoscopic application of a fluid to a cavity or surface containing the defect and the steps of
ii) application of the cells to the defect substantially simultaneously with a support material, the application being performed at the defect covered by the fluid,
iii) mixing of the cells and the supporting material,
iv) solidification of the supporting material so that the defect Is covered by a mixture of cells and support material without any significant amount of fluid, and
v) optionally, removal of the fluid from the cavity or surface by drainage or suction.
3. A method according to item 2, wherein step i) is prior to steps ii)-v).
4. A method according to item 2, wherein the application of the fluid in step i) is substantially simultaneously to the application of the cells in step ii) and the supporting medium in step iii).
5. A method according to item 4, wherein the fluid is a gas.
6. A method according to any of the preceding items, wherein the animal is a mammal such as a human.
7. A method according to any of the preceding items, wherein the defect is a joint or bone defect.
8. A method according to item 7, wherein the defect is a cartilage defect.
9. A method according to the any of the preceding items, wherein the cells are of suitable origine for targeting a suitable tissue, where the visualization is done by an endoscope.
10. A method according to any of the preceding items, wherein the cells are chondrocytes, osteocytes or osteoblasts.
11. A method according to item 10, wherein the cells are chondrocytes.
12. A method according to any of the preceding items, wherein the cells are homologous and/or autologous chondrocytes.
13. A method according to any of items 2, 3, 5-12, wherein the fluid in step i) is a liquid.
14. A method according to item 13, wherein the liquid is a physiologically acceptable aqueous medium selected from the group consisting of sodium chloride solution, Ringer's solution, a cell culture medium, a cell friendly liquid and the like .
15. A method according to any items 2-14, wherein the support material in step ii) is selected from the group consisting of soluble collagens, fibrinogens and aprotinins.
16. A method according to item 15, wherein the support material is applied in the form of an aqueous composition.
17. A method according to item 16, wherein the aqueous composition further comprises one or more adhesion-promoting agents and/or one ore more physiologically acceptable ions such as calcium or magnesium ions.
18. A method according to any of the preceding items, wherein the cells in step ii) are applied in the form of a cell suspension.
19. A method according to item 18, wherein the cells are suspended in a suitable medium such as, e.g., a suitable growth medium optionally comprising one or more growth factors.
20. A method according to item 18 or 19, wherein the cell suspension further comprises one or more coagulating components that initiates the solidification of the support material upon contact between the support material and the coagulating component.
21. A method according to any of items 18-20, wherein the cell suspension further comprises one or more adhesion-promoting agents and/or one or more physiologically acceptable ions such as calcium or magnesium ions.
22. A method according to item 20 or 21, wherein the coagulating component is thrombin or a thrombin-like component.
23. A method according to any of items 2-22, wherein the solidification of the support material is a result of an interaction between the support material and trombin or a trombin-like component and the solidification envelopes the cells in the solidified material.
24. A method according to item 18 or 19, wherein the cell suspension comprises the support material and the method further comprising a step of applying a solution containing a coagulating agent.
25. A method according to any of items 2-23, wherein the mixing of the cells with the support material in step iii) is performed by application the support material and/or the cells under a positive pressure.
26. A method according to item 24, wherein the suspension comprising the cells and the support material is mixed with the coagulating agent by application of the solution containing the coagulating agent under a positive pressure.
27. A kit for use in a method defined in any of the preceding items, the kit comprises two separate containers, the first container comprising the cells and the second container comprising the support material.
28. A method according to item 27, wherein the cells in the first container are in the form of a cell suspension.
29. A kit according to item 28, wherein the cells are suspended in a suitable medium such as, e.g., a suitable growth medium optionally comprising one or more growth factors.
30. A kit according to item 28 or 29, wherein the cell suspension further comprises one or more coagulating components that initiates the solidification of the support material upon contact between the support material and the coagulating component.
31. A kit according to any of items 28-30, wherein the cell suspension further comprises one or more adhesion-promoting agents and/or one or more physiologically acceptable ions such as calcium or magnesium ions.
32. A kit according to item 30 or 31, wherein the coagulating component is thrombin or a thrombin-like component.
33. A kit according to any of items 27-32 further comprising a third container comprising a coagulating component.
34. A kit for use in a method defined in any of the preceding items, the kit comprises two separate containers, the first container comprising the cells and the second container comprising a coagulating agent.
35. A kit according to item 34, wherein the first container comprises the support material.
36. A kit according to item 34 or 35 further comprising a third container comprising the support material.
37. A kit according to any of items 27-36, wherein the kit is in the form of a syringe containing two seperate chambers, the first chamber containing the cells and the second chamber containing the support material or a coagulating agent.
38. A kit according to item 37, wherein the syringe is a Twin syringe or the like.
39. A kit according to any of items 27-38 further comprising instructions for use of the kit.
40. A method according to any of items 1-26 further comprising application of hydroxy apatite e.g. in the form of a hydroxy apatite granulate.
41. Use of hydroxy apatite as culturing medium for cells such as osteoblasts/osteocytes.
42. Use of collagen solutions as culturing medium for the cells to be arthroscopically transplanted.
43. Use of collagen solutions as culturing medium for the cells to be endoscopically transplanted.

The following examples are intended to illustrate the invention without limiting the scope in any way.

### EXAMPLES

### Example 1

A joint derived from an animal, such as for instance a knee joint from a pig or a horse or other animals are subjected to arthroscopy and fluid such as 0.9% sterile saline or a gas is filling up the joint to visualize the area targeted for the arthroscopic repair of the area as for instance a cartilage defect in order to enable the application of the supporting material mixed with the cells in such a manner that the cell/supporting material does not mix with the fluid or gas applied via the arthroscope, and with a retained capability to adhere to the target area.

Alternatively, the cells and or the cells mixed or grown in a hydroxy-apatite suspension (e.g., Bio-Oss), may be delivered through the syringe, and be mixed at the outlet of the syringe into the area to be treated.

### Example 2

In this example, the support material may be placed—under pressure from the other liquid, prior to the implantation of the cells. In this case, the supporting material may be placed below the other liquid for instance kept in place by the pressure exerted via the arthroscope until solidification. The cells are then injected at any time after the placement of the liquid substance or after the solidification has occurred, by simply injecting the cells into the supporting material kept in place by the fluid pressure.

### Example 3

Arthroscopic Autologous Chondrocyte Implantation, Pre-clinical, Combined with Second Fibrin Sealant

First Surgical Arthroscopic Intervention

Five goats are used for the pre-clinical trial. The goats are subjected to two (2) surgical interventions. The first surgical intervention is an arthroscopic harvest of cartilage biopsy from the knee of the goats. The goats are kept at an approved animal facility at Aalborg Sygehus Syd, Denmark. Following anaesthesia, arthroscopy is performed by either a antero-medial or antero-lateral entrance through a small incision. Two additional incisions are prepared on the medial and the lateral side of the knee. Using an elevator, a 50-200 mg cartilage is removed by a sharp elevator, and aseptically transferred to a Transport tube containing sterile Dulbecco MEM/F12 and fetal calf serum. Using a punching instrument, a 0.6 cm (diameter) circular full thickness cartilage lesion is made, to be used for the implantation of cells 3-5 weeks later. The biopsy is transported to Interface Biotech A/S research cell laboratory and is subjected to culturing from explants.

Second Surgical Arthroscopic Intervention

When the chondrocyte culture has been expanded to between 4 and 10 million of cells, which normally takes 3-5 weeks, the cells are returned to the animal facility packed in a Twin-syringe, together with a solution of human recombinant collagen type III plus a compound such as for instance addition of short collagen fibers in one chamber of the Twin-syringe, (short collagen fibers, which has no detrimental effect on cell proliferation, and which, when mixed with collagen gels, have demonstrated permeabilities that were 100 to 1000 times greater compared to when the component only is collagen type III (without short collagen fibers) (Ref. Gentleman E, Nauman, E A, Dee K C and Livesay G A, Tissue Eng. (2004) 10(3-4):421-427) and which accordingly limits any possible contraction caused by the recombinant collagen (when finally mixed with the cells plus thrombin (and calcium clodride from the other chamber of the Twin syringe). The other chamber of the Twin-syringe contains thrombin (and CaCI₂) with a cell concentration of ranging from 1-10 million cells. A line from the tip of one syringe chamber connects with the another line from the other syringe chamber. A blunt needle/catheter at a size of 18 gg is connected to the two lines, so that the mixture containing collagen mixture is mixed with the thrombin and cells when passing through the needle/catheter. The blunt end of the needle/catheter is let—guided by the arthroscope through one of the incisions made for surgical manipulation—to the punched out 0.6 cm (diameter) cartilage defect. The 0,6 cm previously punched out cartilage defect is filled with the collagen mixture—thrombin/cell mixture, which coagulates within 30 to 180 seconds, all depending upon the relative concentration of component involved in the coagulation.

Using another Twin-syringe cell-free fibrin glue or fibrin sealant either as Tisseel (Baxter, Austria), or as Beriplast either in the form of Pantaject® or in the form of Spray-Set (Berijet®), both from Aventis-Behring, is immediately after the application of the cell mixture, applied thoroughly covering the coagulating cell mixture in the defect. The sealant is then allowed to coagulate and the incisions closed. After having completed the second surgical arthroscopic intervention the goat is bandaged (post-operatively) in order to avoid that the cartilage transplant (implant) is tom apart by maximum flexion of the knee joint.

The goat is then subjected to arthroscopic control of the repaired cartilage defect 1 to 2 months after the last arthroscopic treatment. After 6 months post the arthroscopic treatment, the animal is anesthesized and the knee, repaired with the arthroscopic chondrocyte implantation. The lesion will be inspected by the orthopedic surgeon, pictures, videos, taken, densitometric measurement done, and the “treated” area will be harvested and subjected to histological, histochemical, and immune histochemcial analysis.

### Example 4

Arthroscopic Autologous Chondrocyte Implantation, Pre-Clinical, without Second Fibrin Sealant

First Surgical Arhtroscopic Intervention

Five goats are used for the pre-clinical trial. The goats are subjected to two (2) surgical interventions. The first surgical intervention consists of an arthroscopic harvest of cartilage biopsy from the knee of the goats. The goats are kept at an approved animal facility at Aalborg Sygehus Syd, Denmark. Following anaesthesia, arthroscopy is performed by either a antero-medial or antero-lateral entrance through a small incision. Two additional incisions are prepared on the medial and the lateral side of the knee. Using an elevator, a 50-200 mg cartilage is removed by a sharp elevator, and aseptically transferred to a Transport tube containing sterile Dulbecco MEM/F12 and fetal calf serum. Using a punching instrument, a 0.6 cm (diameter) circular full thickness cartilage lesion is made, to be used for the implantation of cells 3-5 weeks later. The biopsy is transported to Interface Biotech A/S research cell laboratory and is subjected to culturing from explants.

Second Surgical Arthroscopic Intervention

When the chondrocyte culture has been expanded to between 4 and 10 million of cells, which normally takes 3-5 weeks, the cells are returned to the animal facility packed in a Twin-syringe, together with a solution of human recombinant collagen type III plus a compound such as for instance addition of short collagen fibers in one chamber of the Twin-syringe, (short collagen fibers, which has no detrimental effect on cell proliferation, and which, when mixed with collagen gels, have demonstrated permeabilities that were 100 to 1000 times greater compared to when the component only is collagen type III (without short collagen fibers) (Ref. Gentleman E, Nauman, E A, Dee K C and Livesay G A, Tissue Eng. (2004) 10(3-4):421-427) and which accordingly limits any possible contraction caused by the recombinant collagen (when finally mixed with the cells plus thrombin (and calcium cloride from the other chamber of the Twin syringe). The other chamber of the Twin-syringe contains thrombin (and CaCI₂) with a cell concentration of ranging from 1-10 million cells. A line from the tip of one syringe chamber connects with the another line from the other syringe chamber. A blunt needle/catheter at a size of 18 gg is connected to the two lines, so that the mixture containing collagen mixture is mixed with the thrombin and cells when passing through the needle/catheter. The blunt end of the needle/catheter is let—guided by the arthroscope through one of the incisions made for surgical manipulation—to the punched out 0.6 cm (diameter) cartilage defect.

The 0.6 cm previously punched out cartilage defect is filled with the collagen mixture—thrombin/cell mixture, which coagulates within 30 to 180 seconds, all depending upon the relative concentration of component involved in the coagulation. No second sealant is used.

After having completed the second surgical arthroscopic intervention the goat is bandaged (post-operatively) in order to avoid that the cartilage transplant (implant) is torn apart by maximum flexion of the knee joint.

The goat is then subjected to arthroscopic control of the repaired cartilage defect 1 to 2 months after the last arthroscopic treatment. After 6 months post the arthroscopic treatment, the animal is anesthesized and the knee, repaired with the arthroscopic chondrocyte implantation. The lesion will be inspected by the orthopedic surgeon, pictures, videos, taken, densitometric measurement done, and the “treated” area will be harvested and subjected to histological, histochemical, and immune histochemcial analysis.

### Example 5

Arthroscopic Autologous Chondrocyte Implantation, Using the Fibrinogen-Thrombin Approach; Pre-Clinical Trial, Combined with Second Fibrin Sealant

First Surgical Arthroscopic Intervention

Five goats are used for the pre-clinical trial. The goats are subjected to two (2) surgical interventions. The first surgical intervention consists of an arthroscopic harvest of cartilage biopsy from the knee of the goats. The goats are kept at an approved animal facility at Aalborg Sygehus Syd, Denmark. Following anaesthesia, arthroscopy is performed by either a antero-medial or antero-lateral entrance through a small incision. Two additional incisions are prepared on the medial and the lateral side of the knee. Using an elevator, a 50-200 mg cartilage is removed by a sharp elevator, and aseptically transferred to a Transport tube containing sterile Dulbecco MEM/F12 and fetal calf serum. Using a plunger, a 0.6 cm (diameter) circular full thickness cartilage lesion is prepared for later use at the time of the cell transplantation (implantation of cells) after 3-5 weeks. The biopsy is transported to Interface Biotech A/S research cell laboratory and is subjected to culturing from explants.

Second Surgical Arthroscopic Intervention

When the chondrocyte culture has been expanded to between 1 and 10 million of cells, which normally takes 3-5 weeks, the cells are returned to the animal facility packed in a Twin-syringe, together with a solution of fibrinogen in one chamber with a cell concentration of 1-10 million cells in 0.5 cc of the fibrinogen solution. The other chamber of the Twin-syringe contains thrombin (and CaCI₂). Alternatively, the fibrinogen solution is in the first chamber and the cells may be combined with thrombin and calcium chloride in the other chamber chamber A line from the tip of one syringe chamber connects with the other line from the other syringe chamber. A needle/catheter at a size of 18 gg is connected to the two lines, so that the mixture containing cells and fibrinogen is mixed with the thrombin when passing through the needle/catheter. The blunt end of the needle/catheter is let—guided by the arthroscope through one of the incisions made for surgical manipulation—to the punched 0.6 cm (diameter) defect.

The 0.6 cm previously punched cartilage defect is filled with the cell-fibrinogen—thrombin mixture, or, alternatively with the fibrinogen—thrombin/cell mixture, which coagulates within 30 to 180 seconds.

Using another Twin-syringe cell-free fibrin glue or fibrin sealant either as Tisseel (Baxter, Austria), or as Beriplast either in the form of Pantaject® or in the form of Spray-Set (Berijet®), both from Aventis-Behring, is immediately after the application of the cell mixture, applied thoroughly covering the coagulating cell mixture in the defect. The sealant is then allowed to coagulate and the incisions closed. After having completed the second surgical arthroscopic intervention the goat is bandaged (post-operatively) in order to avoid that the cartilage transplant (implant) is torn apart by maximum flexion of the knee joint.

The goat is then subjected to arthroscopic control of the repaired cartilage defect 1 to 2 months after the last arthroscopic treatment. After 6 months post the arthroscopic treatment, the animal is anesthesized and the knee, repaired with the arthroscopic chondrocyte implantation. The lesion will be inspected by the orthopedic surgeon, pictures, videos, taken, densitometric measurement done, and the “treated” area will be harvested and subjected to histological, histochemical, and immune histochemcial analysis.

### Example 6

Arthroscopic Autologous Chondrocyte Implantation, Using the Fibrinogen-Thrombin Approach; Pre-Clinical Trial, without Second Fibrin Sealant

First Surgical Arthroscopic Intervention

Five goats are used for the pre-clinical trial. The goats are subjected to two (2) surgical interventions. The first surgical intervention consists of an arthroscopic harvest of cartilage biopsy from the knee of the goats. The goats are kept at an approved animal facility at Aalborg Sygehus Syd, Denmark. Following anaesthesia, arthroscopy is performed by either a antero-medial or antero-lateral entrance through a small incision. Two additional incisions are prepared on the medial and the lateral side of the knee. Using an elevator, a 50-200 mg cartilage is removed by a sharp elevator, and aseptically transferred to a Transport tube containing sterile Dulbecco MEM/F12 and fetal calf serum. Using a plunger, a 0.6 cm (diameter) circular full thickness cartilage lesion is prepared for later use at the time of the cell transplantation (implantation of cells) after 3-5 weeks. The biopsy is transported to Interface Biotech A/S research cell laboratory and is subjected to culturing from explants.

Second Surgical Arthroscopic Intervention

When the chondrocyte culture has been expanded to between 1 and 10 million of cells, which normally takes 3-5 weeks, the cells are returned to the animal facility packed in a Twin-syringe, together with a solution of fibrinogen in one chamber with a cell concentration of 1-10 million cells in 0.5 cc of the fibrinogen solution. The other chamber of the Twin-syringe contains thrombin (and CaCI₂). Alternatively, the fibrinogen solution is in the first chamber and the cells may be combined with thrombin and calcium chloride in the other chamber chamber A line from the tip of one syringe chamber connects with the other line from the other syringe chamber. A needle/catheter at a size of 18 gg is connected to the two lines, so that the mixture containing cells and fibrinogen is mixed with the thrombin when passing through the needle/catheter. The blunt end of the needle/catheter is let—guided by the arthroscope through one of the incisions made for surgical manipulation—to the punched 0.6 cm (diameter) defect.

The 0.6 cm previously punched cartilage defect is filled with the cell-fibrinogen—thrombin mixture, or, alternatively with the fibrinogen—thrombin/cell mixture, which coagulate with 30 to 180 seconds, depending upon the concentration of the individual coagulating components.

After having completed the second surgical arthroscopic intervention the goat is bandaged (post-operatively) in order to avoid that the cartilage transplant (implant) is torn apart by maximum flexion of the knee joint.

The goat is then subjected to arthroscopic control of the repaired cartilage defect 1 to 2 months after the last arthroscopic treatment. After 6 months post the arthroscopic treatment, the animal is anesthesized and the knee, repaired with the arthroscopic chondrocyte implantation. The lesion will be inspected by the orthopedic surgeon, pictures, videos, taken, densitometric measurement done, and the “treated” area will be harvested and subjected to histological, histochemical, and immune histochemcial analysis.

1. An endoscopic method for treating cartilage or bone defects in an animal, said method comprising the steps of:
i) identifying the position of the defect,
ii) applying cells selected from the group consisting of chondrocytes, chondroblasts, osteocytes and osteoblasts and combinations thereof into the cartilage or bone defect.

2. A method according to claim 1 for arthroscopic or endoscopic implantation of homologous or autologous cells into a defect of an animal body, the method comprising a step of
i) arthroscopic or endoscopic application of a fluid to a cavity or surface containing the defect
and the steps of
ii) application of the cells to the defect substantially simultaneously with a support material, the application being performed at the defect covered by the fluid,
iii) mixing of the cells and the supporting material,
iv) solidification of the supporting material so that the defect is covered by a mixture of cells and support material without any significant amount of fluid, and
v) optionally, removal of the fluid from the cavity or surface by drainage or suction.

3. A method according to claim 2, wherein step i) is prior to steps ii)-v).

4. A method according to claim 2, wherein the application of the fluid in step i) is substantially simultaneously to the application of the cells in step ii) and the supporting medium in step iii).

5. A method according to claim 4, wherein the fluid is a gas.

6. A method according to claim 1, wherein the animal is a mammal such as a human.

7. A method according to claim 1, wherein the defect is a joint or bone defect.

8. A method according to claim 7, wherein the defect is a cartilage defect.

9. A method according to claim 1, wherein the cells are of suitable origin for targeting a suitable tissue, where the visualization is done by an endoscope.

10. A method according to claim 1, wherein the cells are chondrocytes, osteocytes or osteoblasts.

11. A method according to claim 10, wherein the cells are chondrocytes.

12. A method according to claim 1, wherein the cells are homologous and/or autologous chondrocytes.

13. A method according to claim 2, wherein the fluid in step i) is a liquid.

14. A method according to claim 13, wherein the liquid is a physiologically acceptable aquous medium selected from the group consisting of sodium chloride solution, Ringer's solution, a cell culture medium, a cell friendly liquid and the like.

15. A method according to claim 2, wherein the support material in step ii) is selected from the group consisting of soluble collagens, fibrinogens and aprotinins.

16. A method according to claim 15, wherein the support material is applied in the form of an aqueous composition.

17. A method according to claim 16, wherein the aqueous composition further comprises one or more adhesion-promoting agents and/or one ore more physiologically acceptable ions such as calcium or magnesium ions.

18. A method according to claim 1, wherein the cells in step ii) are applied in the form of a cell suspension.

19. A method according to claim 18, wherein the cells are suspended in a suitable medium such as, e.g., a suitable growth medium optionally comprising one or more growth factors.

20. A method according to claim 18, wherein the cell suspension further comprises one or more coagulating components that initiates the solidification of the support material upon contact between the support material and the coagulating component.

21. A method according to claim 18, wherein the cell suspension further comprises one or more adhesion-promoting agents and/or one or more physiologically acceptable ions such as calcium or magnesium ions.

22. A method according to claim 2O, wherein the coagulating component is thrombin or a thrombin-like component.

23. A method according to claim 2, wherein the solidification of the support material is a result of an interaction between the support material and trombin or a trombin-like component and the solidification envelopes the cells in the solidified material.

24. A method according to claim 18, wherein the cell suspension comprises the support material and the method further comprising a step of applying a solution containing a coagulating agent.

25. A method according to claim 2, wherein the mixing of the cells with the support material in step iii) is performed by application the support material and/or the cells under a positive pressure.

26. A method according to claim 24, wherein the suspension comprising the cells and the support material is mixed with the coagulating agent by application of the solution containing the coagulating agent under a positive pressure.

27. A kit for use in a method defined in claim 1, the kit comprises two separate containers, the first container comprising the cells and the second container comprising the support material.

28. A method according to claim 27, wherein the cells in the first container are in the form of a cell suspension.

29. A kit according to claim 28, wherein the cells are suspended in a suitable medium.

30. A kit according to claim 28, wherein the cell suspension further comprises one or more coagulating components that initiates the solidification of the support material upon contact between the support material and the coagulating component.

31. A kit according to claim, wherein the cell suspension further comprises one or more adhesion-promoting agents and/or one or more physiologically acceptable ions such as calcium or magnesium ions.

32. A kit according to claim 3O, wherein the coagulating component is thrombin or a thrombin-like component.

33. A kit according to claim 27 further comprising a third container comprising a coagulating component.

34. A kit for use in a method of claim 1, the kit comprises two separate containers, the first container comprising the cells and the second container comprising a coagulating agent.

35. A kit according to claim 34, wherein the first container comprises the support material.

36. A kit according to claim 34 further comprising a third container comprising the support material.

37. A kit according to claim 27, wherein the kit is in the form of a syringe containing two separate chambers, the first chamber containing the cells and the second chamber containing the support material or a coagulating agent.

38. A kit according to claim 37, wherein the syringe is a Twin syringe.

39. A kit according to claim 27, further comprising instructions for use of the kit.

40. A method according to claim 1 further comprising application of hydroxy apatite e.g. in the form of a hydroxy apatite granulate.

41-43. (canceled)

44. A method for culturing cells comprising using a culture medium comprising hydroxy apatite.

45. A method for culturing cells to be arthroscopically transplanted comprising using a culture medium comprising one or more collagen compositions or solutions.

46. A method for culturing cells to be endoscopically transplanted comprising using a culture medium comprising one or more collagen compositions or solutions.
